(19) United States
(12) Patent Application Publication
(10) Pub. No.: US 2016/0120525 A1
(43) Pub. Date: May 5, 2016

(54) Devices and Methods for Closing Tissue Openings

(71) Applicant: ZHIP MediTech, Inc., Hillsborough, CA (US)

(72) Inventors: Ivan P. Zhurylo, Orel (RU); Sergey O. Sablin, Hillsborough, CA (US)

(21) Appl. No.: 14/932,790

(22) Filed: Nov. 4, 2015

Related U.S. Application Data
(60) Provisional application No. 62/075,812, filed on Nov. 5, 2014.

(51) Int. Cl. A61B 17/00

(52) U.S. Cl. CPC A61B 17/0057; A61B 2017/00637; A61B 2017/0065; A61B 17/085

(57) ABSTRACT

A medical device to assist surgical procedures by ensuring a controlled incision and a fast and non-invasive wound closure. The device may comprise two sections, each of which comprises a plurality of plates for adhering the device to the skin. The two sections may straddle either side of an incision and then may be brought together to close the incision. The device may minimize complications associated with wound healing, accelerate the wound healing process, and minimize scar formation.

### CROSS-REFERENCE TO RELATED APPLICATIONS

The present application claims the benefit of U.S. Provisional Application No. 62/075,812, filed on Nov. 5, 2014, the entire disclosure of which is hereby incorporated by reference for all purposes.

### FIELD OF THE INVENTION

The present technology relates generally to medical devices, and, more particularly, to devices and methods for closing surgical incisions and lacerations in tissue.

### BACKGROUND

Effective closure of surgical incisions in an operative patient's tissue (skin, tissue, organs, etc.) is an essential postsurgical process to regain anatomical function and tissue integrity. Common methods for closing tissue openings caused by lacerations or surgical incisions include suturing and stapling. Both of these methods are invasive, which can traumatize and compromise the integrity of the tissue opening and the nutrient blood supply to the healing tissue edges. They cause pain, increase the possibility of infection, inflammation, expose the surgeon and patient to blood-borne diseases, leave behind scars, and require a follow-up visit for suture or staple removal.

Other methods of wound closure include various substances that cover skin edges and hold them adjacent, such as glues and adherent structures, such as tapes and strips. These methods are adequate only for small wounds where skin edges are not widely separated or under tension during closure.

### SUMMARY

The present disclosure is directed to tissue closure devices and methods for their use. An exemplary tissue closure device may comprise a first flexible guide arm having a first base member, a first side member, and a first top member, the first side member comprising a first inner surface. One or more first tissue attachment plates may be coupled to the first guide arm in a generally perpendicular arrangement. A second flexible guide arm may have a second base member, a second side member, and a second top member, the second side member comprising a second inner surface. One or more second tissue attachment plates may be coupled to the second guide arm in a generally perpendicular arrangement. One or more releasable guide arm coupling devices may be in contact with the first and second top members such that the first inner surface is maintained adjacent to the second inner surface along a length of the first and second guide arms.

According to additional exemplary embodiments, the present disclosure may be directed to a tissue closure device. An exemplary tissue closure device may comprise a first flexible guide arm and a second flexible guide arm positioned adjacent to the first guide arm, forming a slot therebetween. A plurality of tissue attachment plates may extend from each of the first and second guide arms. The plurality of tissue attachment plates may be arranged essentially co-planar. Each of the plurality of tissue attachment plates may comprise an adhesive layer.

According to further exemplary embodiments, the present disclosure may be directed to a method for making and closing an incision. An exemplary method may comprise providing a first flexible guide arm releasably coupled to a second flexible guide arm and forming a slot therebetween. One or more tissue attachment plates coupled to each of the first and second guide arms may be provided. An adhesive layer on the one or more tissue attachment plates may be provided, and the adhesive layer may be allowed to couple the one or more tissue attachment plates to tissue. It may be further provided for a tissue cutting instrument to be inserted into the slot. The tissue cutting instrument may be allowed to be moved along the slot to form an incision in the tissue. Decoupling of the first and second guide arms may be provided to allow access to the incision. Recoupling of the first and second guide arms may be provided to close the incision.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

The methods and devices disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 illustrates a perspective view of a tissue closure device according to various embodiments.

FIG. 2 illustrates a cross-sectional view of a tissue attachment plate according to various embodiments.

FIG. 3 illustrates a front perspective view of a tissue closure device according to various embodiments.

FIG. 4 illustrates a perspective view of a tissue closure device according to various embodiments.

FIG. 5 illustrates a front schematic view of a tissue closure device and a scalpel making an incision in tissue according to various embodiments.

FIG. 6 illustrates a top view of a tissue closure device on an open incision according to various embodiments.

FIG. 7 illustrates a perspective view of a tissue closure device being bent according to various embodiments.

FIG. 8A illustrates a perspective view of a tissue closure device with rectangular shaped tissue attachment plates according to various embodiments.

FIG. 8B illustrates a top schematic view of a tissue closure device with a variety of exemplary shapes for tissue attachment plates.

FIG. 9 illustrates a top schematic view of a variety of exemplary vent holes in tissue attachment plates according to various embodiments.

FIG. 10A illustrates a perspective view of an exemplary guide arm coupling device according to various embodiments.

FIG. 10B illustrates a perspective view of an exemplary guide arm coupling device according to various embodiments.

FIG. 10C illustrates a perspective view of an exemplary guide arm coupling device according to various embodiments.

FIG. 11 illustrates a flow diagram of an exemplary method for making and closing an incision according to various embodiments.

### DETAILED DESCRIPTION

While this technology is susceptible of embodiment in many different forms, there is shown in the drawings and will herein be described in detail several specific embodiments with the understanding that the present disclosure is to be considered as an exemplification of the principles of the technology and is not intended to limit the technology to the embodiments illustrated.

The terminology used herein is for the purpose of describing particular embodiments only and is not intended to be limiting of the technology. As used herein, the singular forms “a”, “an” and “the” are intended to include the plural forms as well, unless the context clearly indicates otherwise. It will be further understood that the terms “comprises” and/ or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that like or analogous elements and/or components, referred to herein, may be identified throughout the drawings with like reference characters. It will be further understood that several of the figures are merely schematic representations of the present technology. As such, some of the components may have been distorted from their actual scale for pictorial clarity.

The trend towards minimally invasive wound closure devices and methods is one of the decisive factors affecting the market for surgical incision closure products. Such devices are intended to be non-invasive and provide better approximation of wound edges. However, even recently developed devices and methods do not provide an ideal approximation of wound edges, which extends the healing process and may result in inflammation. They also do not provide a rigid fixation of the wound, which also hinders healing. Also, none of the known methods provide control over the quality of the incision.

The present disclosure is directed to devices and methods for conducting surgical incisions and for non-invasive precision closure of surgical wounds in tissue. The use of various embodiments of the devices and methods disclosed may solve a number of problems associated with the closure of surgical wounds. The device may provide complete and fast wound closure; it may be non-invasive; and it may be suitable for wounds of any size. The use of the device may reduce the chance of inflammation and the healing period of the wound. The use of the device may allow bringing wound edges to a maximum proximity, which positively affects the healing process and minimizes the formation of a scar.

Additionally, various embodiments may provide control over the incision by making an incision between guides of the device. Flexibility of the components of the device may ensure an adequate wound opening required for the surgical procedure. Control over the precision of post-surgical matching of components of the device may be provided in order to ensure an ideal approximation of wound edges. Various embodiments may ensure wound rest during wound healing by maintaining a rigid connection between components of the device in a wound area. An option may be provided through the use of the device to inspect the wound and/or wound drainage during wound healing. Safe removal of the device after wound healing may also be provided.

The present disclosure includes a controlled incision and the ability to bring wound edges to a proximity of their pre-incision position after the surgery. Furthermore, unlike in other methods of wound closure, various embodiments may be applied to the skin before the incision is made and is not removed until the wound is healed. No known wound closure device or method has these features.

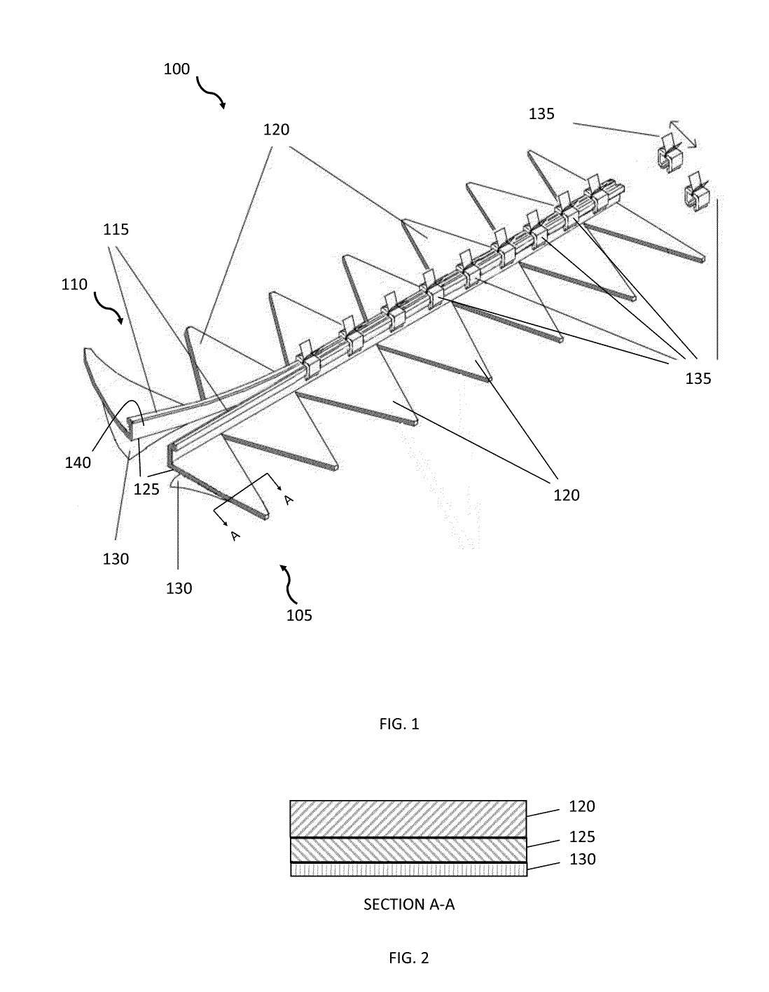

FIGS. 1 through 3 illustrated exemplary embodiments of a tissue closure device 100. The exemplary tissue closure device 100 may comprise a first section 105 and a second section 110. Each of the first and second sections 105, 110 may comprise a semi-rigid guide arm 115, with one or more tissue attachment plates 120 extending from each guide arm 115. In various embodiments, the first and second sections 105, 110 may essentially be mirror images of one another, although this distinction is not required. An exemplary guide arm 115 may comprise a base member 305 and a top member 315 in a spaced apart and generally parallel arrangement with each other. The guide arm 115 may further comprise a side member 310 coupled to both the base member 305 and the top member 315 such that the base member 305, side member 310, and the top member 315 are positioned in a J-shaped or U-shaped arrangement. Each of the side members 310 may further comprise an inner surface 140, such that the inner surfaces 140 of the guide arms 115 of the first and second sections 105, 110 oppose one another as illustrated in the various embodiments of FIG. 1. Further, when one or more guide arm connecting devices 135 are coupled to the guide arms 115, the inner surfaces 140 of each side member 115 may be in releasable contact with one another.

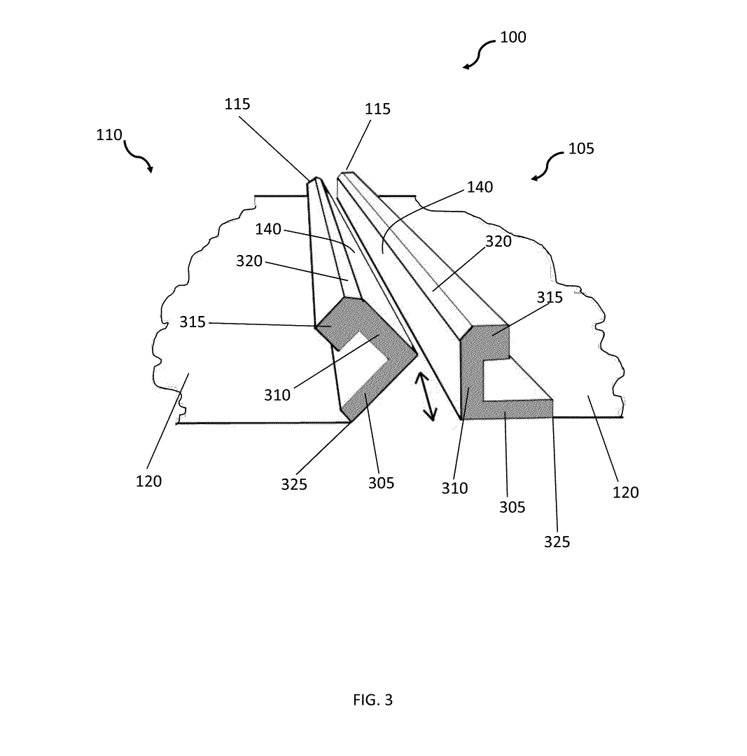

In various embodiments as illustrated in FIG. 1, each of the first and second sections 105, 110 may further comprise a plurality of tissue attachment plates 120. The tissue attachments plates may comprise an extension of the base member 305 of the guide arm 115 (that is, the tissue attachment plates 120 and the base member 305 are a single, continuous component), or the tissue attachment plates 120 may comprise separate components coupled to the base member 305. The tissue attachment plates 120 may be rigid or semi-rigid, and may be constructed of any material suitable for a surgical field, such as but not limited to plastic, rubber, polymer, and the like. As illustrated in FIG. 3, the base member 305 and the tissue attachment plates 120 may be coupled by a hinge 325. The hinge 325 may allow the guide arms 115 to flip at least partially over the tissue attachment plates 120 and away from each other. Positioning the guide arms 115 in this manner may allow improved access to the wound during a surgical procedure, or to inspect the wound for proper healing after a surgical procedure.

FIG. 2 schematically illustrated various embodiments of Section A-A of the tissue attachment plate 120. A bottom surface of the tissue attachment plate 120 may be at least partially coated with an adhesive layer 125. The adhesive layer 125 may in turn be at least partially covered by a protective cover 130 that may be removed just prior to using the tissue closure device 100. The adhesive layer 125 may comprise a temporary or semi-permanent skin bonding agent such as a pressure sensitive adhesive. In some embodiments, the adhesive layer 125 may comprise a skin glue such as but not limited to cyanoacrylates or other acrylic resins, thrombin glue, mixtures of bovine serum albumin (BSA) and glutaraldehyde, and the like. In various embodiments, the adhesive layer 125 may be placed on the tissue attachment plate 120 during manufacture or just prior to use of the tissue closure device 100. In some embodiments, the adhesive layer 125 may first be applied to the tissue, and the tissue attachment plates then placed on the adhesive layer 125.

Some embodiments are further provided with at least one programmed adhesive layer 125. Programmed adhesives may be designed to lose the functionality of the adhesive after a programmed period of time or range of time. Using such adhesive layers 125, a wound may be healed and treated with tissue attachment plates 120 designed to lose their adhesive characteristic with the skin after some period of time, in some cases obviating the need for a trained medical professional to remove the tissue closure device 100.

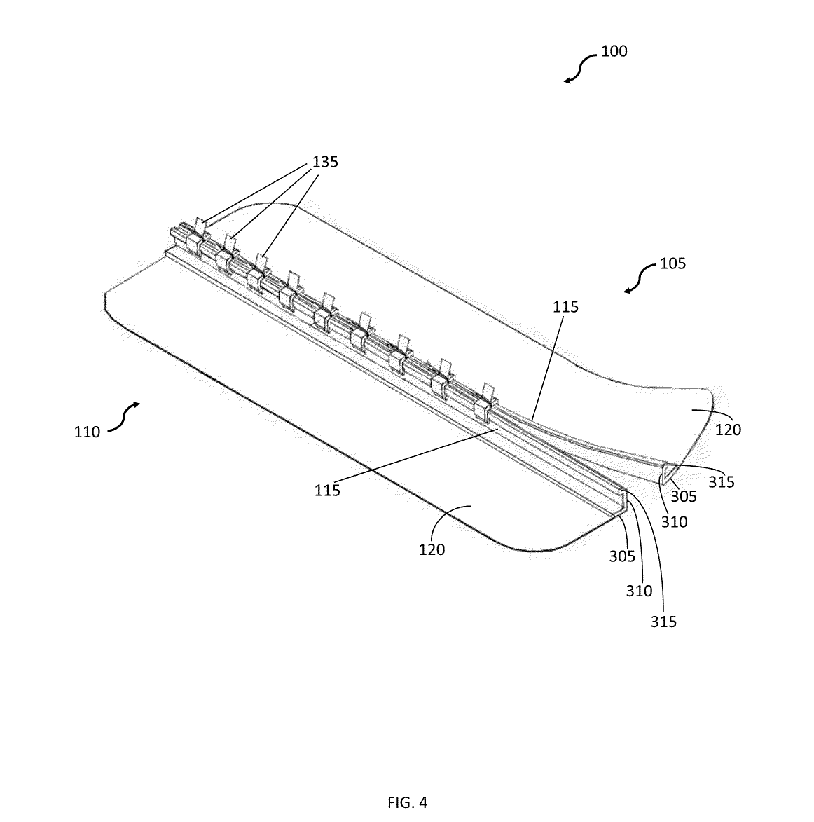

In various embodiments, the tissue attachment plate 120 may comprise a stretchable breathable (porous) medicinal tape. Using a stretchable breathable medicinal tape for the tissue attachment plates 120 may increase the flexibility of the tissue closure device 100 and may allow a wider wound opening, which may be required for certain surgical procedures. FIG. 4 illustrates various embodiments in which each of the first and second sections 105, 110 comprise a single tissue attachment plate 120 comprising stretchable breathable medicinal tape. In some embodiments, the tissue attachment plate 120 may comprise more than one tissue attachment plate 120 comprising medicinal tape.

Various embodiments as illustrated in FIGS. 1 and 4 may further comprise one or more guide arm coupling devices 135. Each guide arm coupling device 135 may simultaneously span across the top members 315 of both guide arms 115 of the first and second sections 105, 110. The guide arm coupling devices 135 may releasably hold the two guide arms 115 together to minimize slippage or other movement of the first and second sections 105, 110 relative to one another. This lack of relative movement may be desirable when placing the tissue closure device 100 on tissue as described further below.

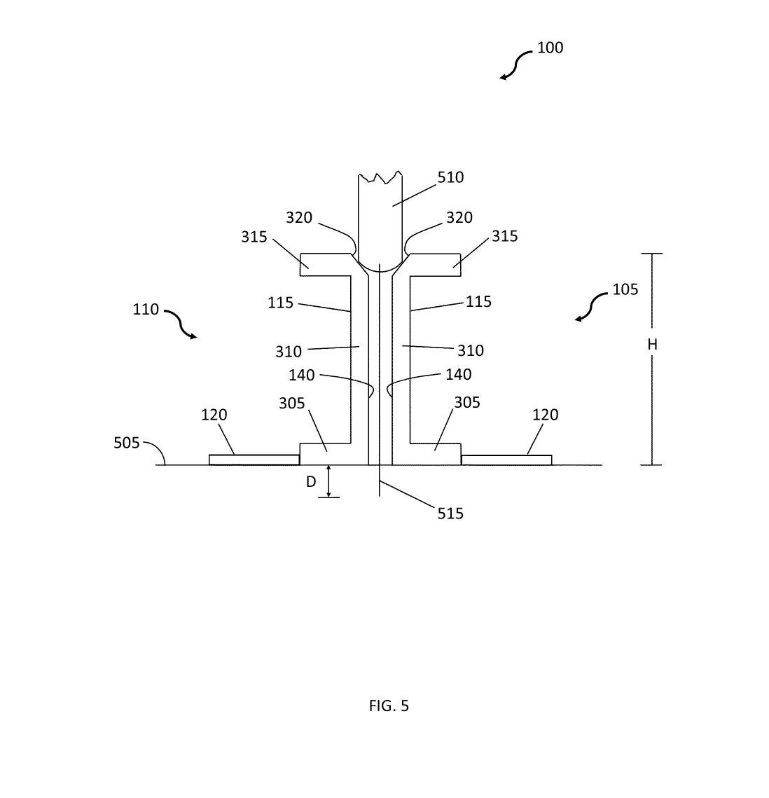

FIG. 5 illustrates an end view of various embodiments of the tissue closure device 100 positioned on a surface of a tissue 505, such as the skin on a human body. Here, the tissue closure device 100 has been placed on the skin 505 and the adhesive layer 125 (not shown) releasably couples the tissue closure device 100 to the skin 505. The guide arm coupling devices 135 have been removed to allow access between the guide arms 115 of the first and second sections 105, 110. An edge where the inner surface 140 of the side member 310 meets the top member 315 may be chamfered, forming a chamfered surface 320. When the first and second section 105, 110 are brought together as shown in FIG. 5, the chamfered edges 320 may form a channel. A surgeon may position a blade 515 of a scalpel 510 between the guide arms 115 until the scalpel 510 contacts the chamfered edges 320 forming the channel. Once the scalpel 510 contacts the chamfered edges 320, the scalpel blade 515 may be positioned a predetermined depth D into the skin 505. Thus, the guide arms 115 may allow the surgeon to make a precise incision in the skin 505 at a controlled depth D. The depth D of the incision may be preselected by specifying a height H of the side members 310 of the guide arms 115. A larger height H may result in a shallower incision, while a smaller height H may result in a deeper incision for a given scalpel 510. The surgeon may then draw the scalpel 510 along the chamfered edges 320 of the guide arms 115 to complete the incision.

Although FIG. 5 illustrates the use of a conventional scalpel 510 to make the incision, any device or method known in the art to create an incision may be used with the tissue closure device 100. Exemplary devices and methods include, but are not limited to, electrosurgical instruments, ultrasound instruments, and the like.

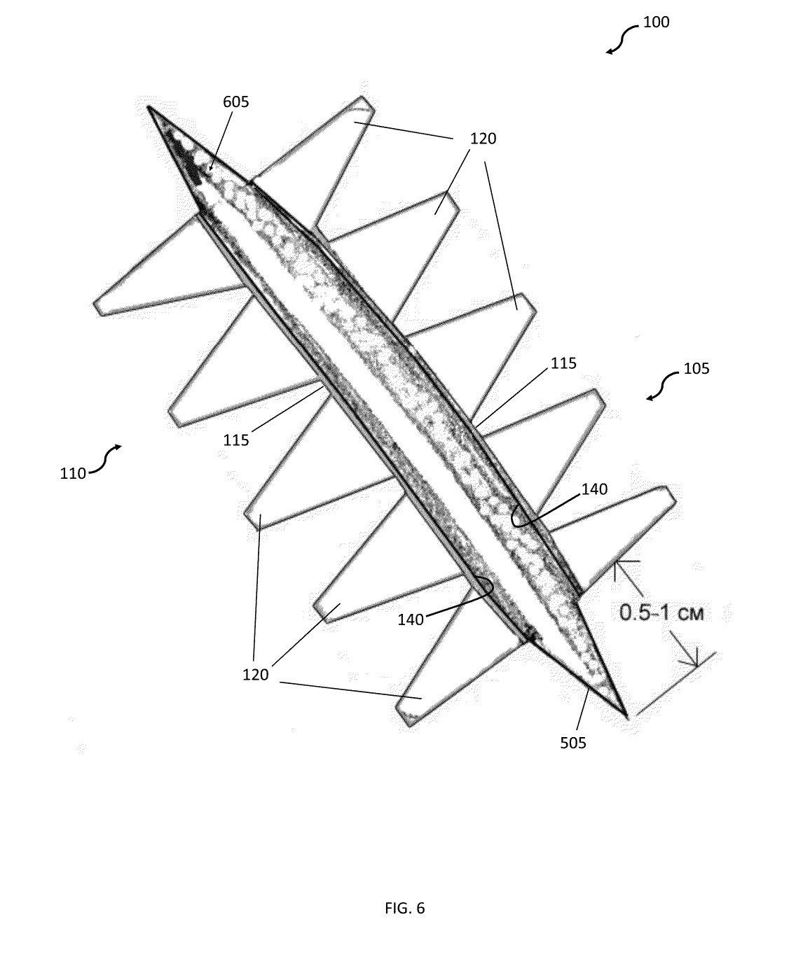

FIG. 6 illustrates an incision 605 in the skin 505 with the tissue closure device 100 in place according to various embodiments. The surgeon may make the incision 605 the approximate length of the tissue closure device 100, or the incision 605 may extend about 0.5 to about 1 cm beyond each end of the tissue closure device 100 to facilitate a wider opening in the incision 605. Because the tissue attachment plates 120 are coupled to the skin 505 by the adhesive layer 125, the inner surfaces 140 of each of the guide arms 115 remain aligned with edges of the skin 505 forming the incision 605. At the end of the surgical procedure the inner surfaces 140 of the guide arms 115 may be brought together, thereby bringing the edges of the skin 505 forming the incision 605 to proximity. One or more of the guide arm coupling devices 135 may be coupled to the top members 315 of the guide arms 115 to hold the guide arms 115 together and seal the incision 605. The number of guide arm coupling devices 135 required to hold the guide arms 115 together may depend on the length of the incision 605 and the degree of the incision 605 opening. Thus, matching incision 605 edges to their position as they were before the surgery may be safely secured in order to provide a wound rest during its healing.

In various embodiments, the guide arms 115 may incorporate an alignment feature to assist the surgeon to precisely realign the two sides of the incision 605 when the incision 605 is closed. The alignment feature may comprise one or more keys that fit into slots. For example, one of the guide arms 115 may comprise one or more keys extending out from the inner surface 140, and the other guide arm 115 may have slots to receive the keys. Since the keys and slots must be aligned in order for the guide arms 115 to be brought together, the original alignment of the skin 505 at the incision 605 may be maintained. In some embodiments, the guide arm coupling devices 135 may comprise clips that are incorporated into the top member 315 of one of the guide arms 115. The coupling clip 135 may have to align with a receiver on the other guide arm 115, thereby properly aligning the incision. Other alignment features as known in the art are within the scope of this disclosure.

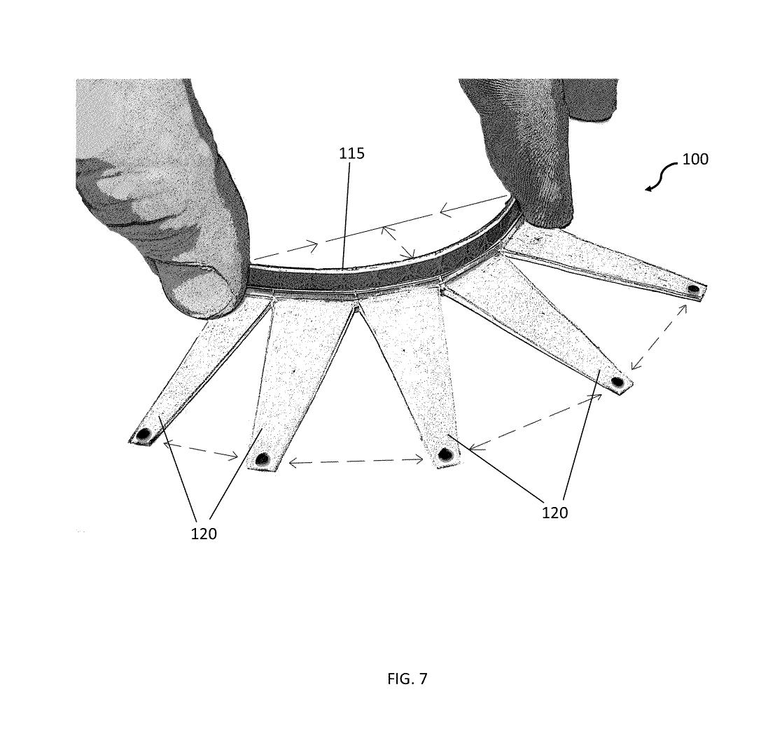

Although FIG. 6 illustrates a relatively straight incision 605, various embodiments may be used to create a curved (simple or complex) incision. Due to the flexible nature of the guide arms 115, the surgeon may bend them prior to coupling the tissue closure device 100 to the skin 505 as illustrated in FIG. 7. Triangular shaped or otherwise spaced apart tissue attachment plates 120 facilitate bending of the guide arms 115 while still allowing the tissue attachment plates 120 to remain generally planar to one another.

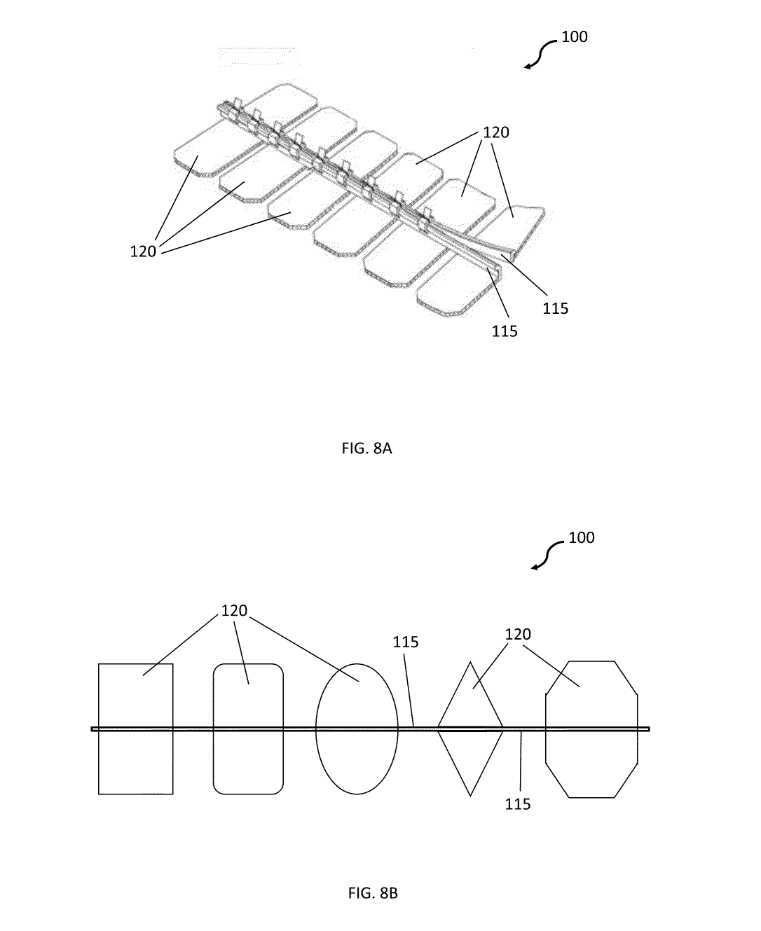

While the preceding figures were based on embodiments comprising triangular shaped tissue attachment plates 120, one skilled in the art will readily recognize that any shape, as well as any number, of tissue attachment plates 120 are within the scope of the present disclosure. For example, FIG. 8A illustrates various embodiments comprising generally rectangular tissue attachment plates 120, with a space between adjoining tissue attachment plates 120 (that is, adjacent tissue attachment plates 120 on the guide arm 115 need not contact one another, although they may contact in some embodiments as illustrated in FIG. 1). FIG. 8B illustrates a variety of exemplary shapes for the tissue attachment plates 120. Although not shown in FIG. 8B, opposing tissue attachment plates 120 may comprise different shapes.

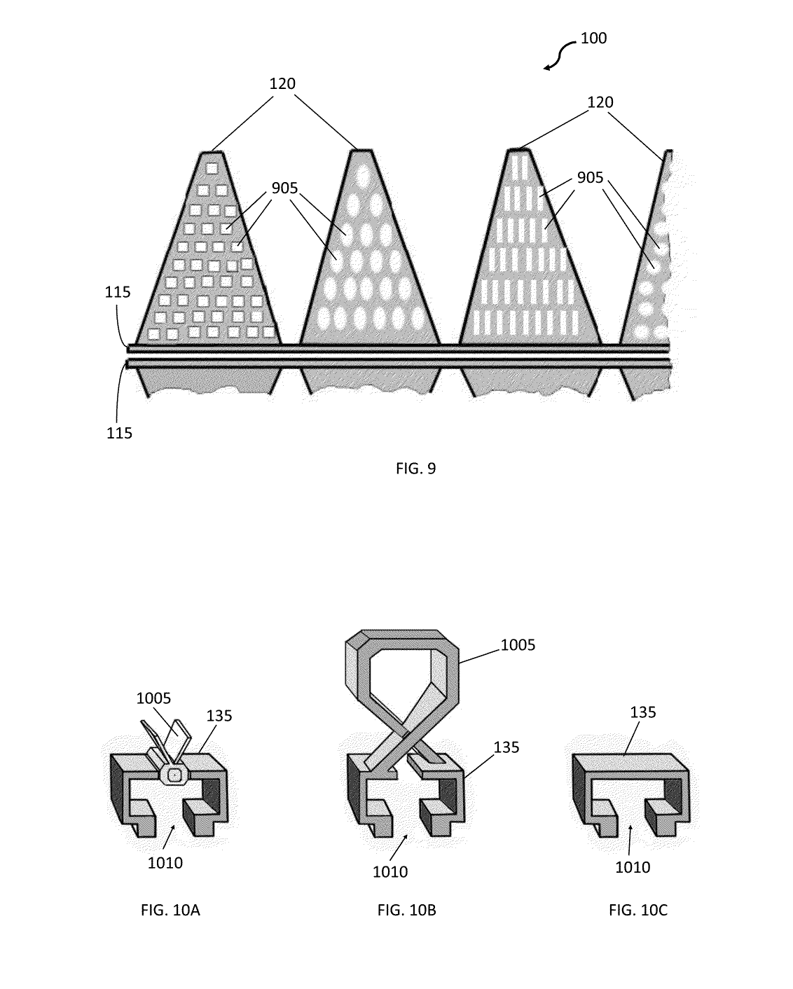

Perforations 905 in the tissue attachment plates 120 may provide for skin breathing, which if not sufficient, may be a cause of negative side effects. The size and the shape of the perforations 905 may be of any desired shape, size, orientation, or number as illustrated in FIG. 9. One skilled in the art will readily recognize that perforations 905 other than those shown in FIG. 9 are with the scope of the present disclosure. The shape of the perforations 905 may be round, elliptic, slit-like, square, trapezoid, multi-faceted, etc. An excessive amount of perforations 905 may reduce the surface area of attachment of the skin attachment plates 120 to the skin 505, thus reducing the reliability of fixation of the tissue closure device 100 on the skin 505.

FIGS. 10A through 10C illustrates exemplary embodiments of the guide arm coupling device 135. The guide arm coupling device 135 may comprise a channel 1010 adapted to fit over both of the guide arm top members 315 when the top members 315 are adjacent to one another such that the inner surfaces 140 are touching. The guide arm coupling devices 135 may further comprise a bending mechanism 1005 that when moved may open the channel 1010 wide enough to slip over the top of the guide arm top members 315 as opposed to slipping the guide arm connecting devices 135 on from the end of the guide arm top members 315.

If it becomes necessary to insert a probe into the incision 605 after the incision 605 has been closed using the tissue closure device 100 or a temporary drainage procedure arises, one or a few of the guide arm coupling devices 135 may be removed or opened to open an inspection area between the guide arms 115. The remainder of the guide arm coupling devices 135 may remain on the guide arms 115 to minimize dislocation of the edges of the incision 605 so that the rest of the incision 605 is not compromised.

Various embodiments provide a number of advantages for closing surgical wounds, include the following. The structure of the device 100 comprising a first and second section 105, 110 may allow a surgeon to conduct a skin incision 605 which is not obscured by any other structural elements. This may keep the incision ends free from obstacles and may allow an adequate opening of the incision 605 for the surgical procedure. The chamfered edges 320 of the guide arms 115 may facilitate a penetration of a scalpel blade 515 between the guide arms 115. The guide arms 115 may provide controlled direction of the incision 605 and precise perpendicular positioning of the scalpel blade 515 to the surface of the skin 505. Secure coupling of the tissue attachment plates 120 to the skin 505 around the incision 605 and orientation of the guide arms 115 to the incision edges provides accurate matching of incision edges during closure. The matched edges of the incision 605 may be fixed by coupling the guide arms 115 by the guide arm coupling devices 135. Removing one or more of the guide arm coupling devices 135 may provide the option of partially opening the tissue closure device 100 for either inspection or for inserting a drainage or probe. The tissue closure device 100 may be readily decoupled from the skin 505 by peeling the tissue attachment plates 120 from the skin 505.

In some embodiments, the tissue closure device 100 may be used for conducting surgical procedures and incision 605 closing on different parts of the body and in a variety of anatomical regions. Limitations on the use of the tissue closure device 100 may be due to a surface configuration of an anatomical region where the surgical procedure is conducted. The higher the curvature of the skin 505 surface, the greater risk of losing adhesion between the tissue attachment plates 120 and the skin 505. The risk of losing adhesion may be higher with increased rigidity of the tissue attachment plates 120. This risk may be reduced by using more flexible materials. For example, a higher level of rigidity of the material of construction of the tissue attachment plates 120 may be used for surgeries in the area of the anterior abdominal wall, the lumbar area, the front and rear areas of the chest, as well as longitudinal sections on the extremities (hips, legs, shoulders and forearms). In such cases, the tissue attachment plates 120 may be of any shape (triangular, square, rectangular, trapezoidal, semicircular, etc.). In anatomic areas of the body with higher curvature of the skin 505 surface, such as scalp, face, neck, as well as the limbs, when transverse directions of incisions is performed, it may be advantageous to use surgical tape types of material for making the tissue attachment plates 120, which could have either scalloped or even edges. The tape material may provide a secure fixation of the tissue closure device 100 on the skin 505 surface, and to more readily allow the guide arms 115 to follow the curvature of the surface. The higher the stiffness of the tissue attachment plates 120, the more wound rest (immobilization) may occur during the postsurgical period, which is critical for a quicker wound healing. The tissue closure device 100 may be used for cosmetic surgeries in delicate anatomical areas, such as eyelids, nose, different face and neck areas, breasts, etc. For cosmetic surgeries the tissue closure device 100 may be manufactured in smaller appropriate sizes.

The tissue closure device 100 may also be used to close an existing incision 605 (or other wound to the skin 505). The first section 105 and the second section 110 may be separated from one another by removing the guide arm coupling devices 135. The guide arm 115 of the first section 105 may be carefully aligned along one side of the incision 605 and then the tissue attachment plates 120 of the first section 105 may be adhered to the skin 505. The guide arm 115 of the second section 110 may be carefully aligned along the other side of the incision 605 and the tissue attachment plates 120 of the second section 110 may be adhered to the skin 505. The two guide arms 115 may be brought together such that their inner surfaces 140 are in contact. The guide arm coupling devices 135 may then be positioned on the guide arms 115 to couple the guide arms 115 together, thereby closing the incision 605.

In some embodiments, the tissue closure device 100 may be manufactured in predetermined sizes, sterilized, and packaged as a ready to use device in the operation room. In other embodiments, the device may be manufactured in pre-ordered sizes and sterilized as needed.

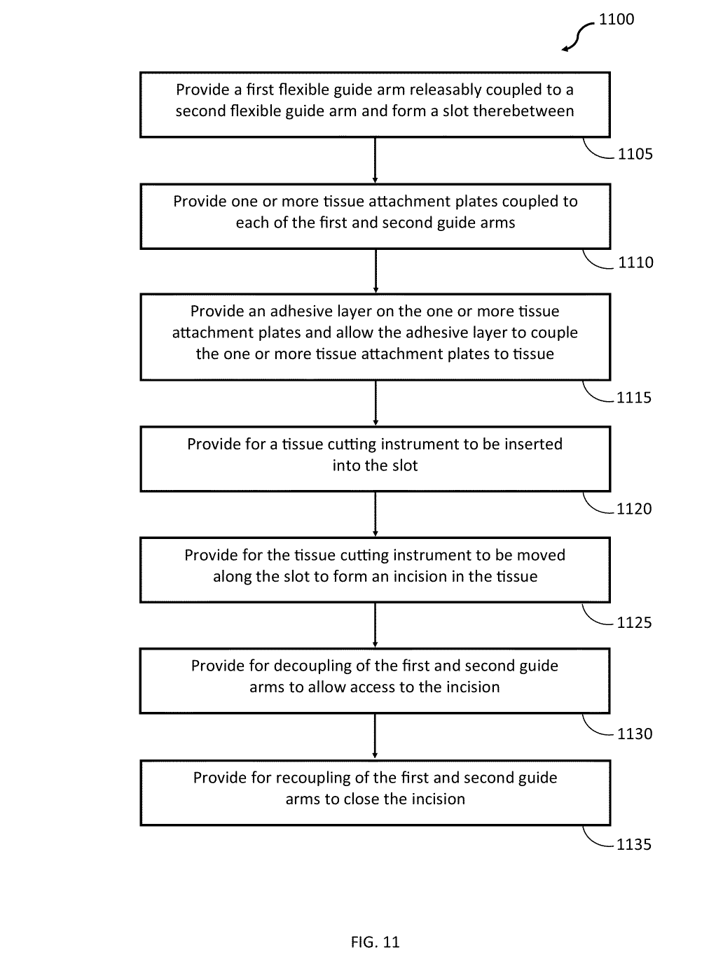

FIG. 11 is a flowchart of an exemplary method for making and closing an incision. At step 1105, a first flexible guide arm 115 may be provided that is releasably coupled to a second flexible guide arm 115, which forms a slot therebetween. One or more tissue attachment plates 120 may be provided at step 1110 that may be coupled to each of the first and second guide arms 115. At step 1115, an adhesive layer 125 may be provided on the one or more tissue attachment plates 120, and the adhesive layer 125 may be allowed to couple the one or more tissue attachment plates 120 to tissue 505. A tissue cutting instrument 510 may be provided at step 1120 that may be inserted into the slot. At step 1125, the tissue cutting instrument 510 may be allowed to be moved along the slot to form an incision 605 in the tissue 505. At step 1130, decoupling of the first and second guide arms 115 may be provided to allow access to the incision 605. Recoupling of the first and second guide arms 115 to close the incision 605 may be provided at step 1135.

While the present technology has been described in connection with a series of preferred embodiments, these descriptions are not intended to limit the scope of the technology to the particular forms set forth herein. It will be further understood that the methods of the technology are not necessarily limited to the discrete steps or the order of the steps described. To the contrary, the present descriptions are intended to cover such alternatives, modifications, and equivalents as may be included within the spirit and scope of the technology as defined by the appended claims and otherwise appreciated by one of ordinary skill in the art.

What is claimed is:

1. A tissue closure device, comprising:
a first flexible guide arm comprising a first base member, a first side member, and a first top member, the first side member comprising a first inner surface;
one or more first tissue attachment plates coupled to the first guide arm in a generally perpendicular arrangement;
a second flexible guide arm comprising a second base member, a second side member, and a second top member, the second side member comprising a second inner surface;
one or more second tissue attachment plates coupled to the second guide arm in a generally perpendicular arrangement; and
one or more releasable guide arm coupling devices in contact with the first and second top members such that the first inner surface is maintained adjacent to the second inner surface along a length of the first and second guide arms.

2. The device of claim 1, wherein the first side member is coupled to both the first base member and the first top member in a generally J-shaped or U-shaped arrangement.

3. The device of claim 1, wherein the second side member is coupled to both the second base member and the second top member in a generally J-shaped or U-shaped arrangement.

4. The device of claim 1, wherein the one or more first tissue attachment plates are coupled to the first base member.

5. The device of claim 1, wherein the one or more second tissue attachment plates are coupled to the second base member.

6. The device of claim 1, wherein the first base member comprises the one or more first tissue attachment plates.

7. The device of claim 1, wherein the second base member comprises the one or more second tissue attachment plates.

8. The device of claim 1, wherein the one or more first tissue attachment plates are generally perpendicular to the first inner surface.

9. The device of claim 1, wherein the one or more second tissue attachment plates are generally perpendicular to the second inner surface.

10. The device of claim 1, wherein the adjacent first and second inner surfaces define a slot therebetween, the slot defining a path for an incision when the tissue closure device is coupled to tissue.

11. The device of claim 1, wherein an edge where the first inner surface and the first top member meet and an edge where the second inner surface and the second top member meet are both chamfered, the chamfered edges forming a channel for guiding a tissue cutting instrument for making an incision when the tissue closure device is coupled to tissue.

12. The device of claim 1, wherein a shape of the one or more first tissue attachment plates is selected from triangular, round, oval, rectangular, or any other regular or irregular shape.

13. The device of claim 1, wherein a shape of the one or more second tissue attachment plates is selected from triangular, round, oval, rectangular, or any other regular or irregular shape.

14. The device of claim 1, wherein a shape of the one or more first tissue attachment plates allows convex or concave bending of the first guide arm without overlap of the one or more first tissue attachment plates.

15. The device of claim 1, wherein a shape of the one or more second tissue attachment plates allows convex or concave bending of the second guide arm without overlap of the one or more second tissue attachment plates.

16. The device of claim 11, wherein each of the first and second side members has a height, the height selected to determine a depth of the incision.

17. The device of claim 1, wherein each of the one or more first and second tissue attachment plates comprises an adhesive layer.

18. The device of claim 1, wherein the one or more first tissue attachment plates comprise porous tape.

19. The device of claim 1, wherein the one or more second tissue attachment plates comprise porous tape.

20. The device of claim 1, further comprising a hinge coupling the one or more first tissue attachment plates to the first guide arm.

21. The device of claim 1, further comprising a hinge coupling the one or more second tissue attachment plates to the second guide arm.

22. A tissue closure device, comprising:
a first flexible guide arm and a second flexible guide arm positioned adjacent to the first guide arm, forming a slot therebetween;
a plurality of tissue attachment plates extending from each of the first and second guide arms, the plurality of tissue attachment plates arranged essentially co-planar; and
an adhesive layer on each of the plurality of tissue attachment plates.

23. The device of claim 22, wherein each of the first and second guide arms comprise a chamfered edge at the slot, the chamfered edges forming a channel for guiding a tissue cutting instrument for making an incision when the tissue closure device is coupled to tissue.

24. The device of claim 22, wherein the slot is adapted to receive a scalpel blade.

25. The device of claim 22, wherein the plurality of tissue attachment plates extend essentially perpendicular from each of the first and second guide arms.

26. The device of claim 22, wherein the plurality of tissue attachment plates comprise a shape to allow convex or concave bending of the first and second guide arms without overlapping any of the plurality of tissue attachment plates.

27. The device of claim 22, wherein each of the first and second guide arms comprise a J-shaped or U-shaped channel.

28. The device of claim 22, wherein the slot defines a path for an incision when the tissue closure device is coupled to tissue.

29. The device of claim 28, wherein each of the first and second guide arms has a height, the height selected to determine a depth of the incision.

30. The device of claim 22, wherein the plurality of tissue attachment plates comprise porous tape.

31. A method for making and closing an incision, comprising:
providing a first flexible guide arm releasably coupled to a second flexible guide arm and forming a slot therebetween;
providing one or more tissue attachment plates coupled to each of the first and second guide arms;
providing an adhesive layer on the one or more tissue attachment plates and allowing the adhesive layer to couple the one or more tissue attachment plates to tissue;
providing for a tissue cutting instrument to be inserted into the slot;
providing for the tissue cutting instrument to be moved along the slot to form an incision in the tissue;
providing for decoupling of the first and second guide arms to allow access to the incision; and
providing for recoupling of the first and second guide arms to close the incision.

32. The method of claim 31, wherein providing one or more tissue attachment plates coupled to each of the first and second guide arms comprises coupling the tissue attachment plates essentially perpendicular to the guide arms.

33. The method of claim 31, wherein forming the slot between the first and second guide arms comprises providing a first inner surface on the first guide arm and a second inner surface on the second guide arm, and placing the first inner surface adjacent to the second inner surface.

34. The method of claim 31, wherein providing one or more tissue attachment plates comprises providing porous tape coupled to each of the first and second guide arms.

35. The method of claim 31, wherein providing for a tissue cutting instrument to be inserted into the slot comprises providing for a scalpel blade to be inserted into the slot.

36. The method of claim 31, wherein providing for recoupling of the first and second guide arms comprises providing a plurality of clips to span across both the first and second guide arms.

37. The method of claim 31, further comprising providing a hinge to couple the one or more tissue attachment plates to each of the first and second guide arms.
